(19) United States
(12) Patent Application Publication
(10) Pub. No.: US 2008/0128367 A1
(43) Pub. Date: Jun. 5, 2008

(54) Method and Disposable Device For Blood Centrifugal Separation

(76) Inventor: Jean-Denis Rochat, Genolier (CH)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 11/815,419

(22) PCT Filed: Feb. 1, 2006
(86) PCT No.: PCT/CH06/00061

§ 371 Date: Aug. 2, 2007

(30) Foreign Application Priority Data
Feb. 3, 2005 (EP) 05405052.1

(51) Int. Cl. B04B 5/04; B04B 7/00; B04B 5/10; A61M 1/36; B01D 17/038

(52) U.S. Cl. 210/782; 494/65; 494/41; 494/31

(57) ABSTRACT

The inventive method for centrifugal separation of a determined volume of physiological liquid, in particular blood, at initial process step, consists in forming a flow of said liquid whose thickness is close to the size of largest particles (L3) contained therein at a volume ratio of <1%, in decelerating said liquid flow in order to increase the thickness thereof and to transfer said largest particles (L3) to the surface of the liquid phase (L1) which is nearest to a centrifugation axis and whose density is the greatest, in arranging, outside of said surface, a dead volume whose capacity is substantially equal to the volume of said largest particles (L3) and in removing a phase (L2) whose density is the lowest.

The present invention relates to a method for the separation of blood by continuous centrifugation and to a disposable device for the continuous separation of a specific volume of blood by centrifugation, comprising a circular centrifugation chamber mounted in rotation about its axis of revolution, an inlet channel for blood to be centrifuged of which the distributing opening is situated close to the bottom of said centrifugation chamber, an outlet passage for at least the constituent separated from said blood having the lower density, of which the collecting opening is situated close to the end of said chamber opposite said bottom, said liquid forming an axial flow against the circular side wall of said chamber between said distributing openings and collecting openings, which is situated in a zone for concentrating said separated constituent so as to withdraw it continuously.

EP 0 257 755 and EP 0 664 159 both relate to a centrifuge bowl for plasmapheresis of the type mentioned above.

When blood is separated with the aid of a device of the type described in EP 0 257 755 or in EP 0 664 159, plasma is substantially obtained that is rich in platelets (PRP) and concentrated red blood cell (RBC). Leucocytes constitute a very small proportion of whole blood, of the order of 0.3% by volume as against 40% for RBCs. Their size may be large, of the order of 12 μm, compared with that of red cells which is of the order of 7 μm, but their density ρ₁=1.08 is very slightly less than that of red cells, ρ_{RBC}=1.095, so that in dynamic sedimentation their sedimentation rate is higher than that of RPCs. On account of this, from the start of centrifugation, they are rapidly precipitated towards the centrifugation wall of the centrifugation chamber. Taking into account the viscosity of RBCs, their proportion and the small respective differences in density of leucocytes and RBCs, leukocytes have considerable difficulty in coming to the surface of the RBC layer during separation of the components of blood by centrifugation, given that leukocytes more often remain trapped in the layer of red cells.

This is the reason why, taking into account their large size, leukocytes are separated by filtering RBCs and PRP after these components have been separated by centrifugation. This supplementary operation thus increases the cost of the blood separation operation and the cost of the disposable device, as well as the loss of RBC in the leukocyte filter.

The object of the present invention is to overcome these disadvantages, at least partially.

To this end, the object of the present invention is first of all a method for separating a specific volume of a physiological liquid, in particular blood, by centrifugation as claimed in claim 1. It also relates to a disposable device for separating a physiological liquid, in particular blood, by centrifugation as claimed in claim 3.

The method and device of the present invention provide a considerable simplification of operations for separating physiological liquids, in particular blood, by enabling leukocytes to be removed from components separated during the operation of separating the liquid by centrifugation.

Advantageously, the ducts for supplying and removing components separated by the device according to the invention are fixed and the two main components RBC and PRP leave the device continuously.

Preferably, the inner face of the side wall of the centrifugation chamber has an annular segment opening out in the direction of axial flow of said liquid in order to produce local acceleration of this flow and a corresponding reduction in the thickness of the layer of said liquid. The object of this flow accelerating zone, bringing about a reduction in thickness, is to enable leukocytes with a density that is very slightly less than that of red cells, but of substantially greater size, to be released from the mass of red cells, so that after the separation zoner when the flow rate falls and the liquid layer increases, leukocytes are situated at the interface between the red cells and the PRP. In addition, this acceleration zone also makes it possible to eject platelets from red cells during concentration, increasing on account of this the platelet yield of the PRP.

The appended drawings illustrate, diagrammatically and by way of example, an embodiment of the method of separation by centrifugation and the disposable device for separating a physiological liquid, in particular blood, that are the subjects of the present invention.

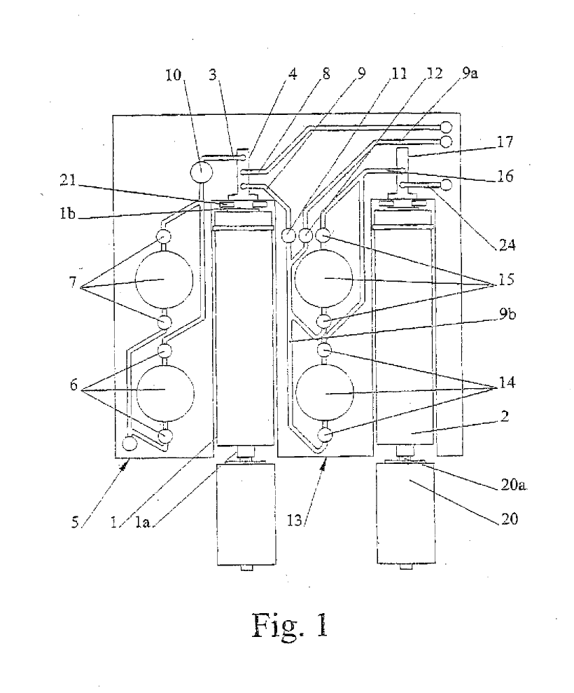

The FIG. 1 is a view in front elevation of a centrifuge separator using this disposable device for implementing this method;

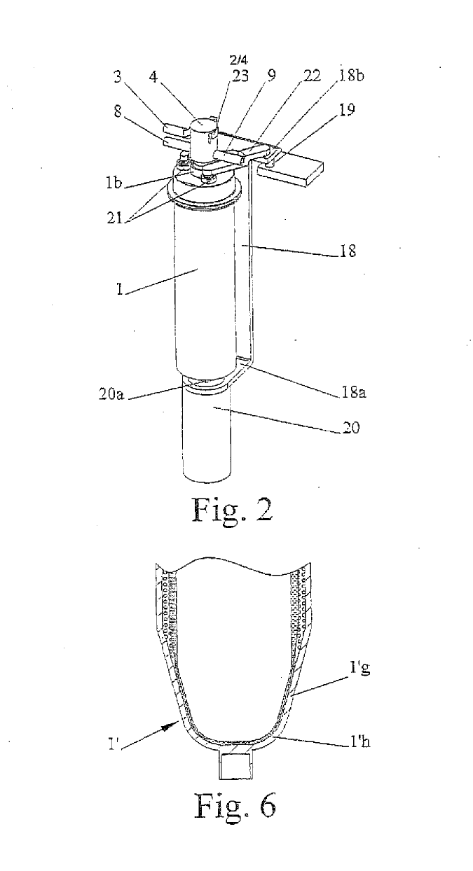

FIG. 2 is a partial perspective view of FIG. 1;

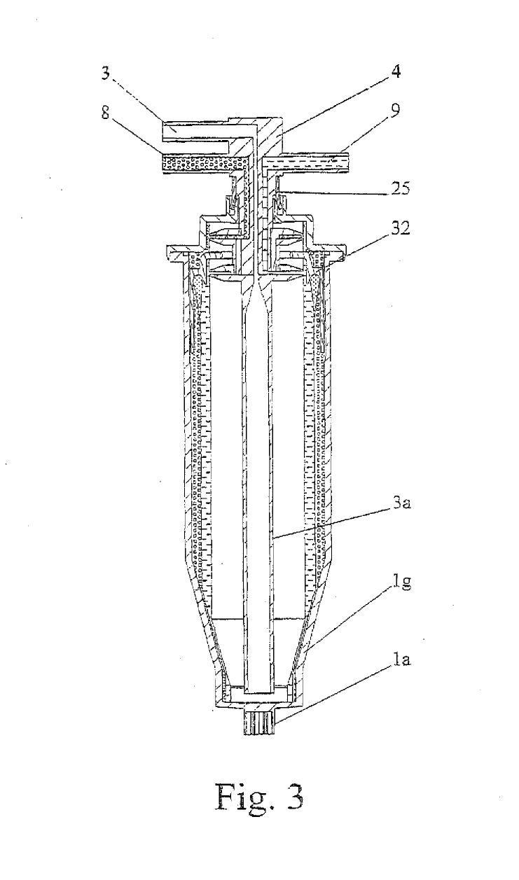

FIG. 3 is a view in axial section of the disposable device of FIGS. 1 and 2;

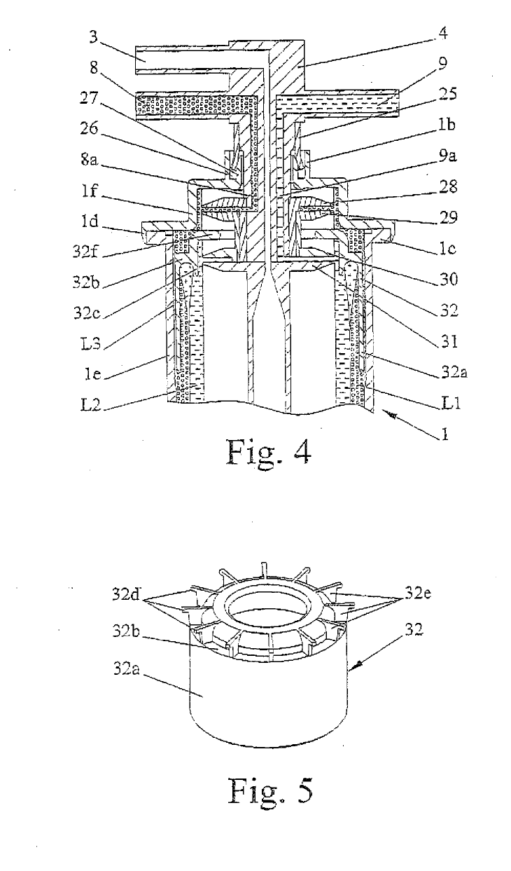

FIG. 4 is a partial enlarged view of FIG. 3;

FIG. 5 is a perspective view of an element of the device of FIGS. 1 and 2;

FIG. 6 is a partial view, in axial section, of a variant of the disposable device according to FIG. 3.

The casing of the centrifuge separator designed to use the device according to the present invention and illustrated diagrammatically by FIG. 1 comprises two elongated centrifugation chambers 1, 2 with a tubular shape. The first tubular centrifugation chamber 1, that is the subject of the present invention, has a supply duct 3, that is connected to a fixed axial inlet and outlet element 4 of the centrifugation chamber 1. This supply duct 3 is connected to a pumping device 5 that comprises two pumps 6 and 7 out of phase by 180° in relation to each other in order to ensure a continuous flow of physiological liquid, in particular blood. An air detector 10 is positioned along the supply duct 3.

Two outlet ducts 8, 9 are connected to the fixed axial element 4, in order to enable the two constituents of physiological liquid with different densities to leave continuously. In the case of blood, the outlet duct 8 is designed for the removal of concentrated RBC red cells and the duct 9 for the removal of plasma rich in PRP platelets. This outlet duct 9 includes a valve 11 and is divided into two branches 9a, 9b. The branch 9a serves to recover the platelet concentrate and is controlled by a valve 12. The valves 11 and 12 operate in an exclusive OR logic either to enable PRP to pass from the chamber 1 to the chamber 2, or to empty the platelet concentration from the chamber 2 to the outlet 9a. The branch 9b serves to lead the PRP to a pumping device 13 comprising two pumps 14 and 15 out of phase by 180° and serving to ensure the continuous supply of the second tubular centrifugation chamber 2 through a supply duct 16 connected to a fixed axial element 17 of the second tubular centrifugation chamber 2. An outlet duct 24 for plasma lean in PPP platelets is also connected to the fixed axial element 17.

FIG. 2 shows the entraining and guiding mode of the substantially tubular centrifugation chamber 1. The assembly of entrainment and guiding elements of the tubular centrifugation chamber is situated on the same support 18 connected to the casing of the centrifugal separator by an anti-vibration suspension 19 of the Silentbloc type. The support 18 has a vertical wall of which the lower end terminates in a horizontal supporting arm 18a to which a drive motor 20 is fixed. The drive shaft 20a of this motor 20 has a polygonal shape, such as a Torx® profile, complementary to an axial recess provided in a small tubular element 1a which projects under the bottom of the tubular centrifugation chamber 1. Coupling between the shaft of the motor 20 and the tubular element 1a should be made with great precision so as to ensure extremely precise guiding of this end of the tubular centrifugation chamber 1.

The upper end of the tubular centrifugation chamber 1 has an axial cylindrical guiding element 1b with a diameter substantially smaller than that of the tubular centrifugation chamber 1, which projects onto its upper face. The cylindrical face of this element 1b is designed to engage with three centering rollers 21. One of these rollers 21 is secured to an arm 22 of which one end is mounted pivotingly upon an upper horizontal part of the support 18. This arm 22 is subjected to the force of a spring (not shown) or any other suitable means, designed to transmit a torque to it tending to make it turn in a clockwise direction, so that it is applied elastically against the cylindrical surface of the axial guiding cylindrical element 1b. On account of this, the tubular centrifugation chamber can be put in place and raised from the support 18 by causing the arm 22 to pivot in an anticlockwise direction. A device for locking the angular position of the arm 22, corresponding to that in which its roller 21 rests against the cylindrical surface of the axial guiding element 1b, is provided so as to prevent having too high a prestress from the spring associated with the arm 22.

The distance between the cylindrical axial guiding element 1b and the upper end of the tubular chamber 1 serves, in cooperation with the centering rollers 21, as an axial stop, preventing the drive shaft 20a of the motor 20 from being uncoupled from the axial recess of the tubular element 1a projecting under the bottom of the tubular chamber 1.

Advantageously, it could also slightly incline the axes of rotation of the guiding rollers 21 by a few angular degrees, <2° in respective planes tangential to the circle that is coaxial with the axis of rotation of the tubular centrifugation chamber 1, passing through the respective axes of rotation of the three rollers, in a chosen direction, according to the direction of rotation of the rollers, in which these induce a force on the tubular chamber 1 directed downwards.

An elastic centering and fixing element 23 of the fixed axial inlet and outlet fixed element 4 of the tubular centrifugation chamber is secured to the upper horizontal part 18b of the support 18. This element 23 has two symmetrical branches, with a semi-circular shape and each of which terminates in a part curved outwards, designed to transmit forces to these elastic arms enabling them to separate from each other when the fixed axial inlet and outlet element 4 is introduced laterally between them.

As can be observed, all the positioning and guiding elements of the fixed and rotating parts of the tubular centrifugation chamber 1 are secured to the support 18, so that precision is a function of the precision of the support 18 itself, that can be produced with very small tolerances, especially as it does not consist of a part that is complicated to produce. Other factors that contribute to guaranteeing a high degree of precision are the relatively large axial distance, due to the elongated tubular shape of this centrifugating chamber, between the lower guide and the upper guide. Finally, the fact of working on a cylindrical guiding surface 1b with a small diameter makes it possible to reduce, on the one hand errors due to contraction of the injected plastic in which the centrifuged chambers 1 and 2 are produced, the contraction being proportional to size, contrary to the case of a machined part, and on the other hand, of out-of-round errors.

This guiding precision for the tubular centrifugation chamber makes it possible to form flows with a very low thickness on the side wall of the centrifugation chamber 1. This therefore makes it possible to have a small volume of liquid remaining in the chamber, which constitutes a factor capable of reducing the risk of hemolysis and platelet activation, this risk certainly being a function of the applied forces, but also of the time during which the components of the blood are subjected to these forces. Thus it is not possible to fix a force threshold, since for a given force the risk of hemolysis can be practically nil for a certain duration, while it can be much greater with the same force but for a substantially longer duration.

Preferably, the tubular centrifugation chamber 1 has a diameter of between 10 and 50 mm, preferably 30 mm, and is driven at a speed of rotation of between 5,000 and 100,000 rpm, so that the tangential velocity to which the liquid is subjected preferably does not exceed 26 m/s. The axial length of the tubular centrifugation chamber 1 advantageously lies between 40 and 200 mm, preferably 90 mm. Such parameters make it possible to provide a liquid flow rate of between 20 and 400 ml/min (in particular for dialysis), preferably 100 ml/min, corresponding to a dwell time for the liquid of 0.5 to 60 s, preferably 5 s in the tubular chamber.

We will now examine in greater detail the design of the tubular centrifugation chamber 1 designed to be associated with the centrifugal separator which has just been described. It can be stated here that all that has been explained in the previous description, as regards dimensions, drive, positioning and guiding of the tubular centrifugation chamber 1, also applies to the tubular centrifugation chamber 2. On the other hand, since the latter only has one outlet 24 for the PPP, it has a simpler design internally than the tubular chamber 1.

As illustrated in FIG. 4, the tubular chamber 1 is made from two parts, the actual tubular chamber 1e and a closing element 1f, both of which end in annular assembling collars 1c, 1d respectively, welded together. The inner space of the tubular part 1e is delimited by the substantially cylindrical wall of this chamber. Close to the bottom of the tubular chamber 1e, its cylindrical side wall has a conical segment 1g (FIG. 3), the function of which will be explained hereinafter.

The fixed axial inlet and outlet element 4 enters this tubular chamber 1 through an axial opening provided in the center of the cylindrical axial guiding element 1b. Leaktightness between this axial opening secured to the centrifugation chamber 1 and the fixed axial element 4 is achieved by means of a tubular seal 25 of which one segment is fixed to a cylindrical portion of this fixed axial inlet and outlet element 4 while another segment is introduced into an annular space 26 of the cylindrical axial guiding element 1b and rests on a convex surface of the tubular wall 27 separating the axial opening traversing the cylindrical axial guiding element 1b from the annular space 26. This seal serves to preserve the sterility of the liquid contained in the centrifuge chamber. As illustrated in this FIG. 4, the part of the tubular seal 25 that rests on the tubular wall 27 is subject to a slight radial deformation in order to ensure leaktightness.

It can be noted that the diameter on which the tubular seal rubs is small and is preferably <10 mm, so that heating is limited to acceptable values. It can also be noted, from the aforementioned possible dimensions given for the tubular centrifuge chamber 1, that the axial distance between the upper centering and guiding means 21 and the lower centering and guiding means 20a of this chamber 1 is five times greater than the diameter of the cylindrical axial guiding element 1b. Taking into account the precision with which the tubular chamber 1 is guided and the precision that relative positioning of the fixed axial inlet and outlet element 4 can attain, the seal has practically nothing to do to compensate for a lack of concentricity of the tubular chamber 1 in rotation, as is the case in devices known in the prior art operating in semi-continuous flow. This also contributes to a reduction in the heating of the rotating tubular seal 25 and therefore makes it possible to increase the speed of rotation of the tubular centrifugation chamber 1.

The fixed axial inlet and outlet element 4 has a tubular part 3a which extends the supply duct 3 connected to this fixed axial element 4 until in proximity to the bottom of the tubular centrifuge element chamber 1 so as to lead there the blood or other physiological fluid to be separated.

Each of the outlet ducts 8 and 9 connected to the fixed axial inlet and outlet element 4 has an axial element 8a, 9a respectively which penetrates into the tubular chamber and emerges in the part of the fixed axial inlet and outlet element 4 which is situated in the vicinity of the upper end of the tubular centrifugation chamber 1. The collecting end of each of these outlet ducts 8a, 9a is formed of a circular slot. Each of these slots is provided between two disks 28, 29 and 30, 31 respectively, secured to the fixed inlet and outlet axial element 4.

The diameters of these four disks 28 to 31 are preferably substantially identical. The circular collecting openings provided between the disks 28, 29, and 30, 31 respectively are separated from each other via a tubular dam 32 illustrated separately in FIG. 5. It has a tubular wall 32a that is concentric and parallel to the lateral wall of the centrifugation chamber 1e. As can be noted in particular in FIG. 4, the radial separation between this tubular wall 32a and the lateral wall of the tubular chamber 1e, as well as the thickness of this tubular wall 32a, are chosen so that this tubular wall 32a is situated entirely within the thickness formed by the L1 phase of the liquid separated by centrifugation having the higher density, corresponding to RECs. The end of this tubular wall 32a furthest from the bottom of the centrifugation chamber 1, has an annular part 32b that closes in the direction of the fixed axial part 4, in the space situated between the disks 29 and 30.

This annular part 32b has an inner annular rim 32c that extends in the direction of the bottom of the centrifugation chamber 1. The diameter of this annular rim 32c is chosen so as to be situated within the thickness formed by the L2 phase of the liquid separated by centrifugation having the smallest density corresponding to the PRP.

On account of this, leukocytes that are in the vicinity of the interface of the L1, L2 phases of the liquid separated by centrifugation, have only one possibility, that of being deposited at the bottom of the annular storage space provided between the tubular wall 32a of the dam 32 and the inner annular rim 32c. These leukocytes L3 accumulate while pushing back the RBCs progressively towards the open end of the dam 32. The volume of the annular space thus provided between the tubular wall 32a and the annular rim 32c is chosen so as to contain at least the volume of leukocytes contained in a specific volume of blood to be centrifuged, for example 450 ml, which is the usual volume of blood taken from a donor, this volume being slightly variable from one individual to another.

As can be noted, the cylindrical portion formed of the annular rim 32c is situated facing the circular collecting opening provided between the disks 30 and 31, in this way isolating this opening from the liquid phases other than the L2 phase intended to be aspirated by this circular collecting opening. This therefore prevents the risk of backflow produced by this aspiration.

The two collecting openings provided respectively between the disks 28, 29 and 30, 31 should be separate so as to enable them to have substantially the same diameters. To this end, the diameter of the inner rim of the part 32f extending radially towards the center of said tubular chamber 1 should be less than those of the disks 28 to 31.

Attachment of the dam 32 is obtained by clamping an annular part 32d between the assembly collars 1c, 1d. This annular part 32d is connected to the actual tubular barrier by arms 32e (FIG. 5) that provide between them openings for the passage of RBCs towards the circular collecting opening provided between the disks 28 and 29.

As can be noted, the diameter of the side wall of the closing element 1f of the tubular chamber 1 is less than that of the side wall of the actual tubular chamber 1e on account of the fact that the tubular barrier 32 is entirely housed within the part 1e of this chamber 1. On account of this, the volume of the RBC immobilized within the centrifugation chamber 1 is reduced.

The role of the conical part 1g (FIG. 3) of the tubular chamber 1 is to reduce locally the thickness of the flow of liquid to be centrifuged by accelerating its flow. By virtue of this truncated conical part 1g where the thickness of the liquid layer is very small, its thickness being close to the size of the leukocytes that often have difficulty emerging from the layer of red cells by reason of their very similar density, of their size that is substantially greater than that of the red cells, and of the viscosity of the latter, these leukocytes no longer have to pass through a relatively large thickness of red cells, so that, when the thickness of the liquid layer increases, once the liquid is in the cylindrical tubular zone, under the effect of the centrifugal force that is exerted on the axial tubular flow of the liquid, the leukocytes remain at the interface which forms between the RBCs and the PRP.

This conical part 1g also has the effect of ejecting the platelets from the red cells during concentration, which makes it possible to increase the platelet yield of the PRP.

When this flow advances in the direction of the circular collecting openings of the outlet ducts 8 and 9, entrained by the PRP, the interface between the RBC and PRP phases enters inside the dam 32 where the leukocytes are trapped in the annular storage zone delimited between the tubular wall 32a and the annular rim 32c.

FIG. 6 illustrates a variant of the shape of the bottom of the tubular centrifugation chamber 1. The bottom of this chamber 1′ is connected to the conical part 1′g by a rounded annular surface 1′h. The role of this surface 1′h is to reduce the transition between the radial flow of the liquid and its axial flow, so as to reduce the risk of hemolysis. At the limit, in the case of a large diameter centrifugation chamber, as is the case in the majority of these, the rounded surface 1′h could have a sufficiently large radius to enable the conical surface 1′g to be replaced, given that this rounded surface 1′h would enable the same objective to be achieved, namely acceleration of the flow and localized thinning of the thickness of the layer.

It should be noted that in all cases, thinning of the layer of the liquid flow, intended to prevent leukocytes being trapped under the RBC layers, requires sufficiently precise guiding of the centrifugation chamber, as the design of the embodiments of the chamber previously described and its variants permit. Indeed, if the precision of this axial guiding of the chamber were to be less than the thickness of the liquid layer thinned to a thickness close to the size of the leukocytes, off-centering of the centrifuge chamber would then not make it possible to obtain a continuous thinned annular or tubular liquid flow layer.

1. A method for the continuous separation of a specific volume of blood by centrifugation, wherein a tubular layer of blood with axial flow subjected to the centrifugal force is supplied in a continuous flow and that, in the initial stage of the centrifugation process, an annular segment is formed in which said tubular layer opens out in the direction of its axial flow so as to increase its speed and to reduce its thickness to a size close to the size of the leukocytes, a tubular layer is then formed with a constant diameter so as to slow the axial flow rate of said tubular layer and to increase its thickness in order to lead the leukocytes to the interface between the phase (L1) of the blood whose density is highest, and that (L2) of which the density is lowest, a dead volume is provided in the vicinity of said interface that is open in the direction of axial flow, with a capacity substantially equal to the volume of said leukocytes contained in said specific volume of blood, and at least the phase (L2) of the blood whose density is lowest is removed.

2. The method as claimed in claim 1, according to which the phase (L1) of the blood having the highest density is also removed.

3. A disposable device for the continuous separation of a specific volume of blood by centrifugation, comprising a circular centrifugation chamber mounted in rotation about its axis of revolution, an inlet channel for blood to be centrifuged of which the distributing opening is situated close to the bottom of said centrifugation chamber, an outlet passage for at least the separated constituent (L2) of said blood having the lowest density, of which the circular collecting opening is situated close to the end of said chamber opposite said bottom, this circular collecting opening being situated in a zone for concentrating said separated constituent so as to withdraw it continuously, characterized in that said chamber has a tubular wall that extends between the distributing opening and the circular collecting opening and that serves as a guide for the axial flow of the blood between these two openings, a tubular dam, concentric with said tubular wall and extending between this tubular wall and said circular collecting opening for removing the phase (L2) of the separated blood having the lowest density and in that an annular rim situated between the tubular dam and the circular collecting opening provides an annular storage space open toward the bottom of this chamber, for collecting the leukocytes (L3).

4. The device as claimed in claim 3, in which the inner limit of said annular storage pocket has a
circular rim that is situated around said circular collecting opening for said phase (L2) having the lower density.

5. The device as claimed in claim 3, in which the length of said tubular centrifugation chamber is greater than its diameter.

6. The device as claimed in claim 3, comprising a fixed axial inlet and outlet element about the axis of which said centrifugation chamber made of plastic is mounted in rotation, a seal rotating between said fixed axial element and said centrifugation chamber, said fixed axial inlet and outlet element having a second outlet passage for at least a second of the separated constituents, of which the collecting opening is situated, in relation to the circular collecting opening for the phase (L2) of the separated blood having the lower density, at an axial distance extending away from the bottom of the centrifugation chamber, the two collecting openings being separated from each other by said tubular dam.

7. The device as claimed in claim 3, in which the inner face of the side wall of said chamber has an annular segment opening out in the direction of axial flow of said liquid in order to produce local acceleration of this flow and a corresponding reduction in the thickness of the layer of said liquid.

8. The device as claimed in claim 7, in which said annular segment opening out in the direction of axial flow of said liquid is situated in the vicinity of the bottom of said chamber.

9. The device as claimed in claim 6, in which the end of said tubular centrifugation chamber opposite its bottom has a cylindrical narrowing through which said fixed axial element passes and in which said rotating seal is positioned.

10. The device as claimed in claim 9, in which the outer surface of said cylindrical narrowing is collecting opening is situated in the zone for concentrating at least one of the separated constituents (L2) having the lower density, is connected to a second centrifugation chamber.

11. The device as claimed in claim 3, in which said fixed outlet duct of which the collecting opening is situated in the zone for concentrating at least one of the separated constituents (L2) having the lower density, is connected to a second centrifugation chamber.

12. The device as claimed in claim 6, in which the collecting openings of said outlet passages are two circular openings with the same diameters, the diameter of the inner rim of said part of said dam extending radially towards the center of said tubular chamber-M being less than that of said collecting openings.

13. The device as claimed in claim 1, in which the bottom of said chamber is connected to its side centrifugation wall by a rounded annular surface.
